(19) United States
(12) Patent Application Publication
(10) Pub. No.: US 2026/0049365 A1
(43) Pub. Date: Feb. 19, 2026

(54) METHOD AND SYSTEM FOR ASSESSING CROP GERMPLASM RESOURCE BASED ON PRIORI KNOWLEDGE

(71) Applicant: China National Rice Research Institute, Hangzhou City (CN)

(72) Inventors: Mengchen Zhang, Hangzhou City (CN); Yaolong Yang, Hangzhou City (CN); Xinghua Wei, Hangzhou City (CN); Yuanyuan Zhang, Hangzhou City (CN); Qun Xu, Hangzhou City (CN); Yue Feng, Hangzhou City (CN)

(21) Appl. No.: 19/052,804

(22) Filed: Feb. 13, 2025

(30) Foreign Application Priority Data
Aug. 13, 2024 (CN) 202411105836.8

(51) Int. Cl. C12Q 1/6895; C12Q 1/6874; G16B 20/00

(52) U.S. Cl. CPC C12Q 1/6895; C12Q 1/6874; G16B 20/00; C12Q 2600/13

(57) ABSTRACT

Provided area a method and system for assessing a crop germplasm resource based on priori knowledge. The method includes: collecting genomic data of a germplasm resource to be measured and performing variation detection thereon to obtain whole-genome genetic variation information of the germplasm resource to be measured; determining variation scores of phenotypic characters of the germplasm resource to be measured according to the whole-genome genetic variation information and a priori knowledge base of crop character variations; and determining a comprehensive variation score of the germplasm resource to be measured based on the variation scores of the phenotypic characters of the germplasm resource to be measured. The method of the present application can rapidly assess the scores of the phenotypic characters of the germplasm resource to be measured according to the genotype data thereof and calculate the comprehensive score in combination with the scores of the phenotypic characters.

### CROSS REFERENCE TO RELATED APPLICATION

This patent application claims the benefit and priority of Chinese Patent Application No. 2024111058368, filed with the China National Intellectual Property Administration on Aug. 13, 2024, the disclosure of which is incorporated by reference herein in its entirety as part of the present application.

### TECHNICAL FIELD

The present disclosure relates to the technical field of assessment of crop germplasm resources, and in particular, to a method and system for assessing a crop germplasm resource based on priori knowledge.

### BACKGROUND

Agricultural germplasm resources are strategic resources for guaranteeing the food safety and the supply of important agricultural products, and are the material basis for original innovations in the seed industry. Protecting and making good use of germplasm resources is an essential condition for developing the sustainable seed industry. Every upgrading of crop varieties in history is accompanied by the discovery and utilization of breakthrough germplasm resources. With the global warming and the frequent outbreak of natural disasters (such as drought and high temperature) and various plant diseases and insect pests, the food safety and the sustainable development of crop production have been seriously affected. Also, with the raising of the people's living standard and the transformation of the production mode, more strict requirements are increasingly put forward on present and future crop varieties and genetic resources. The breeding industry has an urgent need for genetic resources with a clear genetic background, prominent breeding characters, and reliable comprehensive assessment results to carry out variety improvements meeting market requirements. Therefore, studying germplasm resource assessment techniques, and discovering, sharing, and utilizing germplasm resources having an important application value are the focuses of the current resource studies.

For the germplasm resource studies, phenotype is a key point for domesticating and utilizing a crop germplasm resource by humans and is jointly decided by genotype and environment. Genotype is the direct carrier of resource value. Identifying the contribution of genotype to phenotype is the key point for assessing the potential of the germplasm resource. A batch of available excellent resources has been discovered through germplasm resource assessment with phenotype identification as a major method for a long time. However, traditional identification and assessment techniques still have prominent problems such as long cycle, high difficulty, and low throughput. Therefore, how to efficiently and rapidly assess phenotypic scores of a certain cultivar of crop germplasm resources is a technical problem to be urgently solved in the art.

### SUMMARY

An objective of the present disclosure is to provide a method and system for assessing a crop germplasm resource based on priori knowledge that can rapidly and efficiently realize the phenotypic character assessment of the crop germplasm resource.

To achieve the above objective, the present disclosure provides the following technical solutions.

In a first aspect, the present disclosure provides a method for assessing a crop germplasm resource based on priori knowledge, including the following steps:
collecting genomic data of a germplasm resource to be measured, where the genomic data of the germplasm resource to be measured includes gene data of genetic loci;
performing variation detection on the genomic data of the germplasm resource to be measured to obtain whole-genome genetic variation information of the germplasm resource to be measured;
determining variation scores of phenotypic characters of the germplasm resource to be measured according to the whole-genome genetic variation information and a priori knowledge base of crop character variations, where the priori knowledge base of crop character variations includes weights of genetic loci decisive for variations of different phenotypic characters in genomic data of a same type of germplasm resources; and
determining a comprehensive variation score of the germplasm resource to be measured based on the variation scores of the phenotypic characters of the germplasm resource to be measured.

Optionally; the genomic data of the germplasm resource to be measured is collected by any one of resequencing, targeted sequencing, or a gene chip.

Optionally, the priori knowledge base of crop character variations is established by the following steps:
obtaining genomic data of a plurality of known germplasm resources, where the plurality of known germplasm resources and the germplasm resource to be measured are a same type of germplasm resources;
analyzing and determining genetic loci decisive for variations of different phenotypic characters according to the genomic data of the plurality of known germplasm resources to obtain variation locus sets of the phenotypic characters;
for any genetic locus in the variation locus set of any phenotypic character, determining a weight of the genetic locus according to an effect size of the genetic locus on the variation of the phenotypic character; and
establishing the priori knowledge base of crop character variations according to the weights of the genetic loci.

Optionally, the determining variation scores of phenotypic characters of the germplasm resource to be measured according to the whole-genome genetic variation information and a priori knowledge base of crop character variations specifically includes the following steps:
for any phenotypic character, extracting variation data of the corresponding genetic loci from the whole-genome genetic variation information according to the variation locus set of the phenotypic character;
for any genetic locus in the variation locus set of the phenotypic character, determining a variation effect type of the genetic locus according to the variation data of the genetic locus, where the variation effect type includes a favorable allelic variation and other allelic variations;
scoring the genetic loci according to the variation effect types of the genetic loci, respectively, to obtain scores of the genetic loci; and
calculating the variation score of the phenotypic character according to the scores of the genetic loci and the weights of the genetic loci of the phenotypic character.

Optionally; the variation score of the phenotypic character is calculated by the following formula:

$$S = 10 * {\sum }_{k = 1}^{m} {a}_{k} * {w}_{k} ;$$

where S represents a variation score of a single phenotypic character; m represents a number of genetic loci in the variation locus set of the phenotypic character; k represents a mark number of a genetic locus in the variation locus set of the phenotypic character; aₖ represents a score of a k^{th} genetic locus; and wₖ represents a weight of the k^{th} genetic locus.

Optionally; the scoring the genetic loci according to the variation effect types of the genetic loci, respectively; to obtain scores of the genetic loci specifically includes the following steps:
for any genetic locus, if the variation effect type of the genetic locus is the favorable allelic variation, scoring the genetic locus as 1; otherwise, scoring the genetic locus as 0.

Optionally; the phenotypic characters include a plant type, a yield, quality, resistance to insects and diseases, and stress tolerance.

Optionally; the method for assessing a crop germplasm resource based on priori knowledge further includes the following step:
plotting and visually presenting an assessment result graph of the crop germplasm resource based on the variation scores of the phenotypic characters of the germplasm resource to be measured.

Optionally; the determining a comprehensive variation score of the germplasm resource to be measured based on the variation scores of the phenotypic characters of the germplasm resource to be measured specifically includes: averaging the variation scores of the phenotypic characters of the germplasm resource to be measured to determine the comprehensive variation score of the germplasm resource to be measured.

In a second aspect, the present disclosure provides a system for assessing a crop germplasm resource based on priori knowledge, including the following modules:
a genomic data obtaining module configured to collect genomic data of a germplasm resource to be measured, where the genomic data of the germplasm resource to be measured includes gene data of genetic loci;
a genomic data variation detection module configured to perform variation detection on the genomic data of the germplasm resource to be measured to obtain whole-genome genetic variation information of the germplasm resource to be measured;
a phenotypic character scoring module configured to determine variation scores of phenotypic characters according to the whole-genome genetic variation information and a priori knowledge base of crop character variations, where the priori knowledge base of crop character variations includes weights of genetic loci decisive for variations of different phenotypic characters in genomic data of a same type of germplasm resources; and
a comprehensive variation scoring module configured to determine a comprehensive variation score of the germplasm resource to be measured based on the variation scores of the phenotypic characters.

According to specific embodiments provided in the present disclosure, present disclosure has the following technical effects:

The present disclosure provides a method and system for assessing a crop germplasm resource based on priori knowledge. The method includes: collecting genomic data of a germplasm resource to be measured and performing variation detection thereon to obtain whole-genome genetic variation information of the germplasm resource to be measured: determining variation scores of phenotypic characters of the germplasm resource to be measured according to the whole-genome genetic variation information and a priori knowledge base of crop character variations; and determining a comprehensive variation score of the germplasm resource to be measured based on the variation scores of the phenotypic characters of the germplasm resource to be measured. The method of the present disclosure can rapidly assess the scores of the phenotypic characters of the germplasm resource to be measured according to the genotype data thereof and calculate the comprehensive score in combination with the scores of the phenotypic characters. The method is applied to the field of assessment of crop germplasm resources and can greatly improve the assessment efficiency on the phenotypic characters of germplasm resources and save the assessment cost, thereby better realizing efficient discovery and utilization of the germplasm resources.

### BRIEF DESCRIPTION OF THE DRAWINGS

To describe the technical solutions in the embodiments of the present disclosure or in the related art more clearly, the following briefly describes the accompanying drawings required for describing the embodiments or the related art. Apparently; the accompanying drawings in the following description show some embodiments of the present disclosure, and a person of ordinary skill in the art may still derive other accompanying drawings from these accompanying drawings without creative efforts.

FIG. 1 is a flowchart of a method for assessing a crop germplasm resource based on priori knowledge provided by an embodiment of the present disclosure;

FIG. 2 is a flowchart of step S3 in a method for assessing a crop germplasm resource based on priori knowledge provided by an embodiment of the present disclosure;

FIG. 3 is a flowchart of establishing a priori knowledge base of crop character variations in a method for assessing a crop germplasm resource based on priori knowledge provided by an embodiment of the present disclosure;

FIG. 4 is a schematic diagram of a germplasm resource assessment result graph in a method for assessing a crop germplasm resource based on priori knowledge provided by another embodiment of the present disclosure;

FIG. 5 is a schematic diagram of functional modules of a system for assessing a crop germplasm resource based on priori knowledge provided by another embodiment of the present disclosure; and

FIG. 6 is a schematic structural diagram of a computer device provided by another embodiment of the present disclosure.

### DETAILED DESCRIPTION OF THE EMBODIMENTS

The technical solutions in the embodiments of the present disclosure will be described clearly and completely below with reference to the accompanying drawings in the embodiments of the present disclosure. Apparently, the described embodiments are merely some rather than all of the embodiments of the present disclosure. All other embodiments derived from the embodiments in the present disclosure by those of ordinary skill in the art without creative efforts should fall within the protection scope of the present disclosure.

To make the above objective, features, and advantages of the present disclosure more obvious and easier to understand, the present disclosure will be further described in detail with reference to the accompanying drawings and specific implementations.

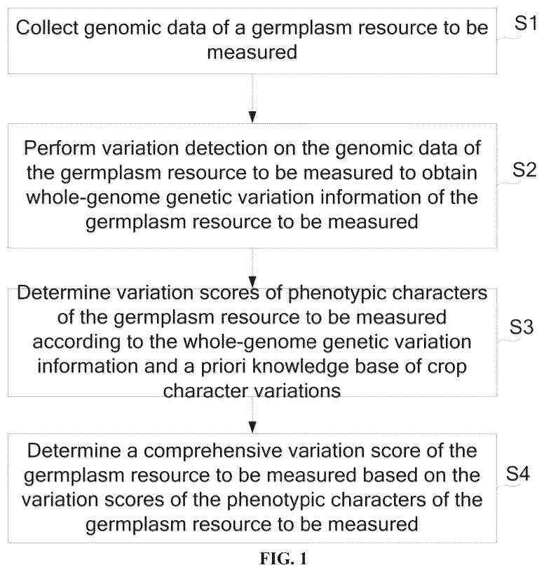

In an exemplary embodiment, as shown in FIG. 1, there is provided a method for assessing a crop germplasm resource based on priori knowledge, including the following steps.

In step S1, genomic data of a germplasm resource to be measured is collected, where the genomic data of the germplasm resource to be measured includes gene data of genetic loci.

In step S2, variation detection is performed on the genomic data of the germplasm resource to be measured to obtain whole-genome genetic variation information of the germplasm resource to be measured.

In step S3, variation scores of phenotypic characters of the germplasm resource to be measured are determined according to the whole-genome genetic variation information and a priori knowledge base of crop character variations, where the priori knowledge base of crop character variations includes weights of genetic loci decisive for variations of different phenotypic characters in genomic data of a same type of germplasm resources. The phenotypic characters include a plant type, a yield, quality, resistance to insects and diseases, and stress tolerance.

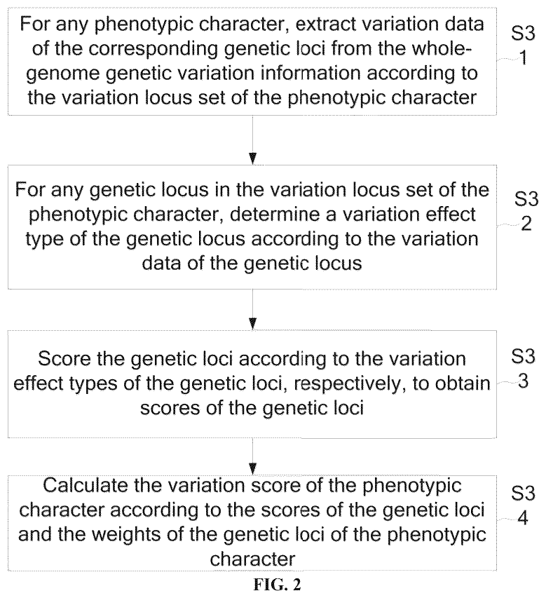

In this embodiment, as shown in the flowchart of FIG. 2, step S3 specifically includes the following steps.

In step S31, for any phenotypic character, variation data of the corresponding genetic loci is extracted from the whole-genome genetic variation information according to the variation locus set of the phenotypic character.

In step S32, for any genetic locus in the variation locus set of the phenotypic character, a variation effect type of the genetic locus is determined according to the variation data of the genetic locus, where the variation effect type includes a favorable allelic variation and other allelic variations.

In step S33, the genetic loci are scored according to the variation effect types of the genetic loci, respectively, to obtain scores of the genetic loci. Specifically, for any genetic locus, if the variation effect type of the genetic locus is the favorable allelic variation, the genetic locus is scored as 1; otherwise, the genetic locus is scored as 0.

In step S34, the variation score of the phenotypic character is calculated according to the scores of the genetic loci and the weights of the genetic loci of the phenotypic character. The variation score of the phenotypic character is calculated by the following formula:

$$S = 10 * {\sum }_{k = 1}^{m} {a}_{k} * {w}_{k} ;$$

where S represents a variation score of a single phenotypic character; m represents a number of genetic loci in the variation locus set of the phenotypic character; k represents a mark number of a genetic locus in the variation locus set of the phenotypic character; aₖ represents a score of a k^{th} genetic locus; and wₖ represents a weight of the k^{th} genetic locus.

In step S4, a comprehensive variation score of the germplasm resource to be measured is determined based on the variation scores of the phenotypic characters of the germplasm resource to be measured. Specifically, the variation scores of the phenotypic characters of the germplasm resource to be measured are averaged to determine the comprehensive variation score of the germplasm resource to be measured.

In an exemplary embodiment of the present disclosure, the genomic data of the germplasm resource to be measured may be collected by any one of resequencing, targeted sequencing, or a gene chip.

In an exemplary embodiment of the present disclosure, the method for assessing a crop germplasm resource based on priori knowledge further includes the following step.

In step S5, an assessment result graph of the crop germplasm resource is plotted based on the variation scores of the phenotypic characters of the germplasm resource to be measured and visually presented.

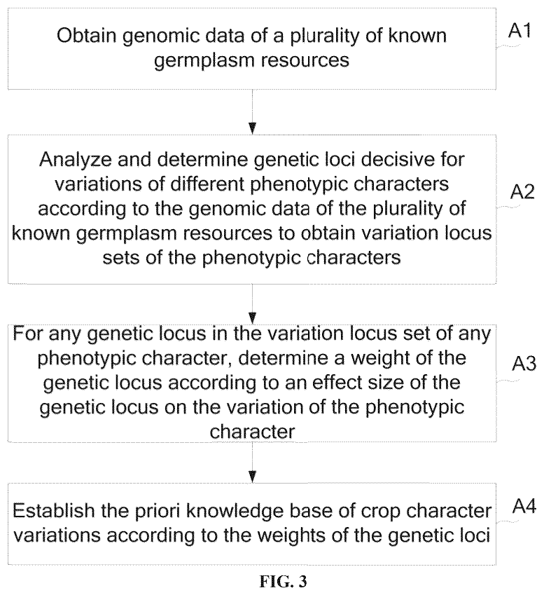

In another exemplary embodiment of the present disclosure, the priori knowledge base of crop character variations needs to be established through the following flow before the above steps S1 to S4 or steps S1 to S5 are performed. As shown in the flowchart of FIG. 3, the following steps are included.

In step A1, genomic data of a plurality of known germplasm resources is obtained, where the plurality of known germplasm resources and the germplasm resource to be measured are a same type of germplasm resources. The above data may be obtained by consulting functional gene research papers of corresponding crops and related databases.

In step A2, genetic loci decisive for variations of different phenotypic characters are analyzed and determined according to the genomic data of the plurality of known germplasm resources to obtain variation locus sets of the phenotypic characters. The genetic loci decisive for variations include a single base nucleotide variation, a deletion/insertion variation, and other molecular marker variations, and their locations may be within genes and in upstream and downstream intervals thereof.

In step A3, for any genetic locus in the variation locus set of any phenotypic character, a weight of the genetic locus is determined according to an effect size of the genetic locus on the variation of the phenotypic character.

In step A4, the priori knowledge base of crop character variations is established according to the weights of the genetic loci.

Next, in a specific embodiment, for the crop rice, a priori knowledge base of crop character variations is established through the following flow; and the rice germplasm resource “ZG9” is assessed by using the priori knowledge base of crop character variations. Specific steps are as follows.

Control genes of agronomic phenotypic characters (such as plant type, yield, quality, resistance to insects and diseases, and stress tolerance) of the rice are collected from the functional gene database and the document database of the rice. The control genes and the upstream and downstream intervals thereof are determined by reading document knowledge to decide the variation locus sets of the phenotypic characters, and sequence features of the favorable allelic variations in these variation locus sets are further specified.

For all genetic loci deciding each agronomic phenotypic character, in combination with the effect sizes of the genetic loci on the variation, the weights of the genetic loci in the phenotypic character are manually marked, with a numerical range of 0 to 1 and a weight sum of 1. The variation information of all the phenotypic characters is further gathered to establish the priori knowledge base of the rice.

Subsequently, the rice germplasm resource to be measured is resequenced using the second generation high-throughput sequencing technique, with a sequencing depth greater than 10×. After original sequencing data is obtained, variation detection is performed using a conventional sequencing data analysis method to obtain the whole-genome genetic variations of the germplasm resource to be measured. Specifically, in this embodiment, when the rice germplasm resource “ZG9” is assessed, the conventional DNA extraction technique is adopted to obtain the whole-genome DNA of the tender leaf tissue of the rice germplasm resource “ZG9”. After fluorescent quantitative inspection, a sequencing library having a fragment size of 400 bp is established, and double-ended sequencing is performed on the library using a high-throughput sequencer. About 35.01 million original reads are obtained in total, and a total data size is 4.9 G. The raw data is cleaned and subjected to quality control. Sequence alignment is performed on the quality controlled data using variation detection software to detect variations and generate a genotype file. The sequencing depth of the ZG9 sequencing data is 12.3×, and the genome coverage is 90.06%. The variation data of corresponding locations is extracted for each germplasm resource to be measured according to the locus information provided by the priori knowledge base.

The scoring rule for a single variant genetic locus in the genotype file is as follows: the favorable allelic variation is scored as 1, and other allelic variations are scored as 0. The scoring result of the germplasm resource to be assessed on the phenotypic character is calculated according to the following formula:

$$S = 10 * {\sum }_{k = 1}^{m} {a}_{k} * {w}_{k} ;$$

where S represents a scoring result of the germplasm resource to be measured on a certain phenotypic character; k represents a genetic locus belonging to the phenotypic character; m represents a total number of genetic loci deciding the phenotypic character; a represents a score of a current genetic locus; and w represents a weight of the current genetic locus.

The phenotypic characters of the germplasm resource to be measured are scored according to the above formula, thus sequentially completing assessment on all the phenotypic characters. Meanwhile, a germplasm resource assessment radar graph or other schematic diagrams may also be plotted using a scripting language or other software. For example, in this embodiment, the comprehensive score of the rice germplasm resource “ZG9” is 6.1, and its assessment result graph is as shown in FIG. 4.

Based on the same inventive concept, an embodiment of the present disclosure further provides a system for assessing a crop germplasm resource based on priori knowledge for implementing the method for assessing a crop germplasm resource based on priori knowledge as described above. The implementation solutions to problems provided by the system are similar to those described in the above method. Thus, for specific definitions in one or more embodiments of the system for assessing a crop germplasm resource based on priori knowledge provided below; reference may be made to those provided above in the method for assessing a crop germplasm resource based on priori knowledge, which will not be described here redundantly:

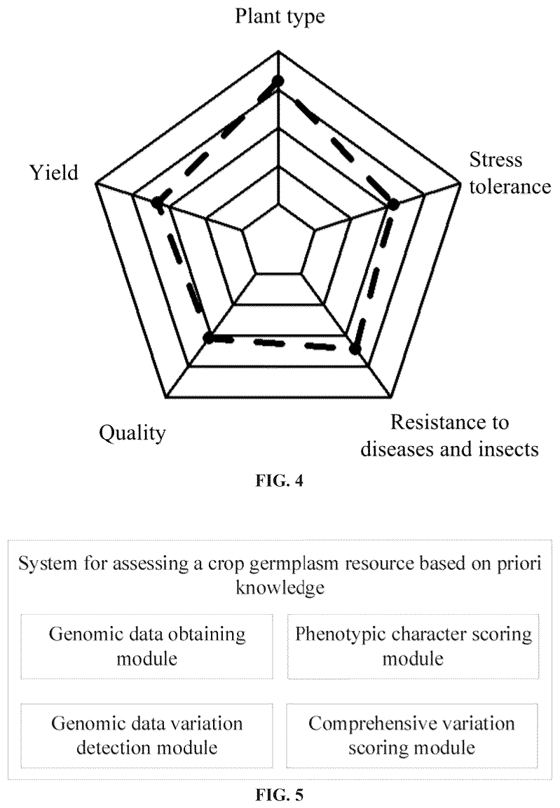

In an exemplary embodiment, as shown in FIG. 5, there is provided a system for assessing a crop germplasm resource based on priori knowledge, including the following modules:
a genomic data obtaining module configured to collect genomic data of a germplasm resource to be measured, where the genomic data of the germplasm resource to be measured includes gene data of genetic loci;
a genomic data variation detection module configured to perform variation detection on the genomic data of the germplasm resource to be measured to obtain whole-genome genetic variation information of the germplasm resource to be measured;
a phenotypic character scoring module configured to determine variation scores of phenotypic characters according to the whole-genome genetic variation information and a priori knowledge base of crop character variations, where the priori knowledge base of crop character variations includes weights of genetic loci decisive for variations of different phenotypic characters in genomic data of a same type of germplasm resources; and
a comprehensive variation scoring module configured to determine a comprehensive variation score of the germplasm resource to be measured based on the variation scores of the phenotypic characters.

Some modules of the system described above may further have sub-units for realizing functions thereof. Of course, the architecture shown in FIG. 5 is only exemplary, and one or at least two components in the system shown in FIG. 5 may be omitted according to actual needs when different functions are achieved.

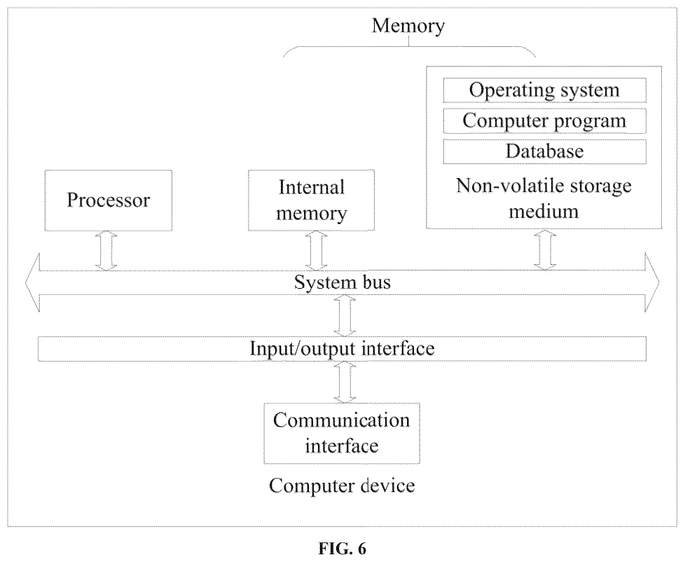

In an exemplary embodiment, a computer device is provided. The computer device may be a server or a terminal, and an internal structure thereof may be as shown in FIG. 6. The computer device includes a processor, a memory, an input/output interface (I/O), and a communication interface. The processor, the memory, and the input/output interface are connected through a system bus, and the communication interface is connected to the system bus through the input/output interface. The processor of the computer device is configured to provide computing and control capabilities. The memory of the computer device includes a nonvolatile storage medium and an internal memory. The non-volatile storage medium stores an operating system, a computer program, and a database. The internal memory provides an environment for operation of the operating system and the computer program in the nonvolatile storage medium. The input/output interface of the computer device is configured to exchange information between the processor and an external device. The communication interface of the computer device is configured to communicate with an external terminal through a network. When the computer program is executed by the processor, the method for assessing a crop germplasm resource based on priori knowledge is implemented.

Those skilled in the art may understand that the structure shown in FIG. 6 is only a block diagram of a part of the structure related to the solutions of the present disclosure and does not constitute a limitation on a computer device to which the solutions of the present disclosure are applied. Specifically: the computer device may include more or less components than those shown in the figure, or combine some components, or have different component arrangements.

It is to be noted that user information (including, but not limited to, user device information, user personal information, and the like) and data (including, but not limited to, data for analysis, data for storage, data for presentation, and the like) involved in the present disclosure are information and data authorized by the user or fully authorized by each party, and relevant data shall be collected, used, and processed according to the related regulations.

Those of ordinary skill in the art may understand that all or some of the procedures in the methods of the above embodiments may be implemented by a computer program instructing related hardware. The computer program may be stored in a nonvolatile computer-readable storage medium. When the computer program is executed, the procedures in the embodiments of the above methods may be performed. Any reference to a memory, a database, or other media used in the embodiments of the present disclosure may include a non-volatile and/or volatile memory. The non-volatile memory may include a read-only memory (ROM), a magnetic tape, a floppy disk, a flash memory, an optical memory; a high-density embedded non-volatile memory, a Resistive Random Access Memory (ReRAM), a Magnetoresistive Random Access Memory (MRAM), a Ferroelectric Random Access Memory (FRAM), a Phase Change Memory (PCM), a graphene memory; and the like. The volatile memory may include a random access memory (RAM) or an external cache memory. As an illustration rather than a limitation, the RAM may be in various forms, such as a static random access memory (SRAM) or a dynamic random access memory (DRAM).

The database in the embodiments of the present disclosure may include at least one of a relational database and a non-relational database. The non-relational database may include a distributed database based on a blockchain, but is not limited thereto. The processor in the embodiments of the present disclosure may be a general processor, a central processor, a graphics processor, a digital signal processor (DSP), a programmable logic device, and a data processing logic device based on quantum computing, but is not limited thereto.

The technical characteristics of the above embodiments can be employed in arbitrary combinations. To provide a concise description of these embodiments, all possible combinations of all the technical characteristics of the above embodiments may not be described: however, these combinations of the technical characteristics should be construed as falling within the scope defined by the specification as long as no contradiction occurs.

Several examples are used herein for illustration of the principles and implementations of this application. The description of the foregoing examples is used to help illustrate the method of this application and the core principles thereof. In addition, those of ordinary skill in the art can make various modifications in terms of specific implementations and scope of application in accordance with the teachings of this application. In conclusion, the content of the present specification shall not be construed as a limitation to this application.

What is claimed is:

1. A method for assessing a crop germplasm resource based on priori knowledge, comprising:
collecting genomic data of a germplasm resource to be measured, wherein the genomic data of the germplasm resource to be measured comprises gene data of genetic loci;
performing variation detection on the genomic data of the germplasm resource to be measured to obtain whole-genome genetic variation information of the germplasm resource to be measured;
determining variation scores of phenotypic characters of the germplasm resource to be measured according to the whole-genome genetic variation information and a priori knowledge base of crop character variations, wherein the priori knowledge base of crop character variations comprises weights of genetic loci decisive for variations of different phenotypic characters in genomic data of a same type of germplasm resources; and
determining a comprehensive variation score of the germplasm resource to be measured based on the variation scores of the phenotypic characters of the germplasm resource to be measured.

2. The method for assessing a crop germplasm resource based on priori knowledge according to claim 1, wherein the genomic data of the germplasm resource to be measured is collected by any one of resequencing, targeted sequencing, or a gene chip.

3. The method for assessing a crop germplasm resource based on priori knowledge according to claim 1, wherein the priori knowledge base of crop character variations is established by the following steps:
obtaining genomic data of a plurality of known germplasm resources, wherein the plurality of known germplasm resources and the germplasm resource to be measured are a same type of germplasm resources;
analyzing and determining genetic loci decisive for variations of different phenotypic characters according to the genomic data of the plurality of known germplasm resources to obtain variation locus sets of the phenotypic characters;
for any genetic locus in the variation locus set of any phenotypic character, determining a weight of the genetic locus according to an effect size of the genetic locus on the variation of the phenotypic character; and
establishing the priori knowledge base of crop character variations according to the weights of the genetic loci.

4. The method for assessing a crop germplasm resource based on priori knowledge according to claim 3, wherein the determining variation scores of phenotypic characters of the germplasm resource to be measured according to the whole-genome genetic variation information and a priori knowledge base of crop character variations specifically comprises:
for any phenotypic character, extracting variation data of the corresponding genetic loci from the whole-genome genetic variation information according to the variation locus set of the phenotypic character;
for any genetic locus in the variation locus set of the phenotypic character, determining a variation effect type of the genetic locus according to the variation data of the genetic locus, wherein the variation effect type comprises a favorable allelic variation and other allelic variations;
scoring the genetic loci according to the variation effect types of the genetic loci, respectively, to obtain scores of the genetic loci; and
calculating the variation score of the phenotypic character according to the scores of the genetic loci and the weights of the genetic loci of the phenotypic character.

5. The method for assessing a crop germplasm resource based on priori knowledge according to claim 4, wherein the variation score of the phenotypic character is calculated by the following formula:
$$S = 10 * {\sum }_{k = 1}^{m} {a}_{k} * {w}_{k} ;$$
wherein S represents a variation score of a single phenotypic character: m represents a number of genetic loci in the variation locus set of the phenotypic character; k represents a mark number of a genetic locus in the variation locus set of the phenotypic character; aₖ represents a score of a k^{th} genetic locus; and wₖ represents a weight of the k^{th} genetic locus.

6. The method for assessing a crop germplasm resource based on priori knowledge according to claim 4, wherein the scoring the genetic loci according to the variation effect types of the genetic loci, respectively, to obtain scores of the genetic loci specifically comprises:
for any genetic locus, if the variation effect type of the genetic locus is the favorable allelic variation, scoring the genetic locus as 1; otherwise, scoring the genetic locus as 0.

7. The method for assessing a crop germplasm resource based on priori knowledge according to claim 1, wherein the phenotypic characters comprise a plant type, a yield, quality, resistance to insects and diseases, and stress tolerance.

8. The method for assessing a crop germplasm resource based on priori knowledge according to claim 1, further comprising:
plotting and visually presenting an assessment result graph of the crop germplasm resource based on the variation scores of the phenotypic characters of the germplasm resource to be measured.

9. The method for assessing a crop germplasm resource based on priori knowledge according to claim 1, wherein the determining a comprehensive variation score of the germplasm resource to be measured based on the variation scores of the phenotypic characters of the germplasm resource to be measured specifically comprises: averaging the variation scores of the phenotypic characters of the germplasm resource to be measured to determine the comprehensive variation score of the germplasm resource to be measured.

10. A system for assessing a crop germplasm resource based on priori knowledge, comprising:
a genomic data obtaining module configured to collect genomic data of a germplasm resource to be measured, wherein the genomic data of the germplasm resource to be measured comprises gene data of genetic loci;
a genomic data variation detection module configured to perform variation detection on the genomic data of the germplasm resource to be measured to obtain whole-genome genetic variation information of the germplasm resource to be measured;
a phenotypic character scoring module configured to determine variation scores of phenotypic characters according to the whole-genome genetic variation information and a priori knowledge base of crop character variations, wherein the priori knowledge base of crop character variations comprises weights of genetic loci decisive for variations of different phenotypic characters in genomic data of a same type of germplasm resources; and
a comprehensive variation scoring module configured to determine a comprehensive variation score of the germplasm resource to be measured based on the variation scores of the phenotypic characters.
